(19) United States
(12) Patent Application Publication
(10) Pub. No.: US 2023/0256164 A1
(43) Pub. Date: Aug. 17, 2023

(54) FLOW STOP ASSEMBLY, PRESSURE PLATE, INFUSION CARTRIDGE AND INFUSION CARTRIDGE ASSEMBLY METHOD

(71) Applicant: TERUMO KABUSHIKI KAISHA, Tokyo (JP)

(72) Inventors: Shun DEMIZU, Hadano-shi (JP); Koshi SANO, Minami-Alps-shi (JP); Hideyuki SATOU, Sendai-shi (JP)

(73) Assignee: TERUMO KABUSHIKI KAISHA, Tokyo (JP)

(21) Appl. No.: 18/137,141

(22) Filed: Apr. 20, 2023

Related U.S. Application Data
(63) Continuation of application No. PCT/JP21/37771, filed on Oct. 12, 2021.

(30) Foreign Application Priority Data
Oct. 28, 2020 (JP) 2020-180658

(51) Int. Cl. A61M 5/168; A61M 5/142

(52) U.S. Cl. CPC A61M 5/16804; A61M 5/14244; A61M 2205/502

(57) ABSTRACT

A flow stop assembly is attachable to an infusion pump and includes: a tube receiving portion comprising a fixing portion configured to be fixed to the infusion pump; and a tube pressing portion coupled to the tube receiving portion via a hinge and rotationally displaceable with respect to the tube receiving portion around a rotation axis. The fixing portion is fixable to the infusion pump in a state in which an infusion tube is inserted between the tube receiving portion and the tube pressing portion.

### CROSS-REFERENCE TO RELATED APPLICATIONS

This application is a bypass continuation of PCT Application No. PCT/JP2021/037771, filed on Oct. 12, 2021, which claims priority to Japanese Application No. JP2020-180658, filed on Oct. 28, 2020. The contents of these applications are incorporated herein by reference in their entireties.

### BACKGROUND

The present disclosure relates to a flow stop assembly, a pressure plate, an infusion cartridge, and a method of assembling an infusion cartridge.

In the related art, there has been known a portable infusion pump that feeds an infusion, such as a drug solution. The portable infusion pump is used in a state in which a cassette accommodating an infusion storage bag is attached. The portable infusion pump feeds the infusion from the infusion storage bag via an infusion tube, and administers the infusion into a living body, such as a patient, while controlling a flow rate.

In the portable infusion pump, it is necessary to provide an anti-free flow (AFF) function in order to prevent a large amount of infusion from being accidentally fed when the infusion tube is detached from the portable infusion pump. The AFF function is a function of blocking the infusion tube to prevent the infusion from being fed when the infusion tube is detached from the portable infusion pump. For example, Japanese Patent No. 6222750B discloses an infusion pump in which the AFF function is implemented by a flow stop assembly.

### SUMMARY

The flow stop assembly disclosed in Japanese Patent No. 6222750B includes two components, i.e., a flow stop blocking arch and a flow stop arm. Therefore, it is necessary to assemble the two components by combining the two components at the time of manufacturing. Accordingly, it is difficult to reduce a manufacturing cost and improve a production efficiency.

An object of the present disclosure is to provide a flow stop assembly, a pressure plate, an infusion cartridge, and a method of assembling an infusion cartridge capable of reducing the number of components.

According to a first aspect of the present disclosure, a flow stop assembly is attachable to an infusion pump and comprises: a tube receiving portion including a fixing portion configured to be fixed to the infusion pump; and a tube pressing portion coupled to the tube receiving portion via a hinge and rotationally displaceable with respect to the tube receiving portion with the hinge as an axis, in which the fixing portion is fixable to the infusion pump in a state in which an infusion tube is inserted between the tube receiving portion and the tube pressing portion.

In one embodiment of the present disclosure, the tube pressing portion is biased so as to selectively block the infusion tube in a state in which the fixing portion is fixed to the infusion pump.

In one embodiment of the present disclosure, the tube receiving portion includes a restriction portion configured to restrict displacement of the infusion tube.

In one embodiment of the present disclosure, the restriction portion is implemented by a groove formed in the tube receiving portion.

According to a second aspect of the present disclosure, a pressure plate for an infusion pump comprises: a flow stop assembly; and a main body portion to which the flow stop assembly is attached, in which the flow stop assembly includes a tube receiving portion including a fixing portion configured to be fixed to the main body portion, and a tube pressing portion coupled to the tube receiving portion via a hinge and rotationally displaceable with respect to the tube receiving portion with the hinge as an axis, and the fixing portion is fixed to the main body portion in a state in which an infusion tube is inserted between the tube receiving portion and the tube pressing portion.

According to a third aspect of the present disclosure, an infusion cartridge for an infusion pump comprises: an accommodation portion configured to accommodate a reservoir bag accommodating an infusion; an infusion tube configured to supply the infusion from the reservoir bag; and a pressure plate attached to the accommodation portion, in which the pressure plate includes a flow stop assembly, the flow stop assembly includes a tube receiving portion and a tube pressing portion coupled to the tube receiving portion via a hinge and rotationally displaceable with respect to the tube receiving portion with the hinge as an axis, and the infusion tube is inserted between the tube receiving portion and the tube pressing portion.

A method of assembling an infusion cartridge according to a fourth aspect of the present disclosure is a method of assembling an infusion cartridge of an infusion pump, the method of assembling an infusion cartridge including: a step of preparing a reservoir bag configured to accommodate an infusion, an accommodation portion configured to accommodate the reservoir bag, and a main body portion and a flow stop assembly forming a pressure plate, in which the flow stop assembly includes a tube receiving portion and a tube pressing portion coupled to the tube receiving portion via a hinge and rotationally displaceable with respect to the tube receiving portion with the hinge as an axis; a step of attaching the flow stop assembly to the main body portion in a state in which an infusion tube connected to the reservoir bag and configured to supply the infusion is inserted between the tube receiving portion and the tube pressing portion; and a step of attaching the pressure plate to the accommodation portion.

A flow stop assembly according to a fifth aspect of the present disclosure is a flow stop assembly attachable to an infusion pump, the flow stop assembly including: a tube receiving portion including a fixing portion configured to be fixed to the infusion pump; a tube pressing portion configured to press an infusion tube by moving toward the tube receiving portion in a state in which the infusion tube is inserted between the tube receiving portion and the tube pressing portion; and a release clip coupled to the tube receiving portion via a hinge and rotationally displaceable with respect to the tube receiving portion with the hinge as an axis, in which the release clip is configured to release, by the rotational displacement, a blocked state of the infusion tube by displacing the tube pressing portion in a direction away from the tube receiving portion.

In one embodiment of the present disclosure, the tube receiving portion and the release clip are torsionally displaceable relative to each other in a rotation axis direction of the hinge.

In one embodiment of the present disclosure, the release clip includes a hook portion at an end portion not coupled to the hinge, and the tube receiving portion includes an engagement portion configured to engage with the hook portion in a state in which the release clip displaces the tube pressing portion in the direction away from the tube receiving portion.

In one embodiment of the present disclosure, the tube receiving portion includes a release portion configured to release an engagement state between the hook portion and the engagement portion in a state in which the release clip is torsionally displaced with respect to the tube receiving portion.

In one embodiment of the present disclosure, the tube pressing portion includes a restriction portion configured to restrict deformation or movement of the hook portion on a side opposite to the engagement portion with the hook portion interposed therebetween in a state in which the hook portion and the engagement portion are engaged with each other.

An infusion cartridge according to a sixth aspect of the present disclosure is an infusion cartridge of an infusion pump, the infusion cartridge including: an accommodation portion configured to accommodate a reservoir bag accommodating an infusion; an infusion tube configured to supply the infusion from the reservoir bag; and a pressure plate attached to the accommodation portion. The pressure plate includes a flow stop assembly. The flow stop assembly includes a tube receiving portion, a tube pressing portion configured to press the infusion tube by moving toward the tube receiving portion in a state in which the infusion tube is inserted between the tube receiving portion and the tube pressing portion, and a release clip coupled to the tube receiving portion via a hinge and rotationally displaceable with respect to the tube receiving portion with the hinge as an axis. The release clip is configured to release, by the rotational displacement, a blocked state of the infusion tube by displacing the tube pressing portion in a direction away from the tube receiving portion.

According to the flow stop assembly, the pressure plate, the infusion cartridge, and the method of assembling an infusion cartridge of the present disclosure, the number of components can be reduced.

### BRIEF DESCRIPTION OF THE DRAWINGS

FIG. 1 is a front view illustrating an infusion pump according to one embodiment of the present disclosure.

FIG. 2 is a perspective view illustrating a pressure plate according to one embodiment of the present disclosure.

FIG. 3 is a perspective view illustrating a flow stop assembly before assembly according to one embodiment of the present disclosure.

FIG. 4 is a view illustrating a state in which a tube pressing portion in FIG. 2 is biased.

FIG. 5 is a view illustrating a state in which a release clip in FIG. 2 is rotationally displaced.

FIG. 6 is a view illustrating one modification of a hinge in FIG. 2.

### DETAILED DESCRIPTION

Hereinafter, embodiments of a flow stop assembly, a pressure plate, an infusion cartridge, and a method of assembling an infusion cartridge according to the present disclosure will be described with reference to the drawings. In the drawings, common members and portions are denoted by the same reference numerals.

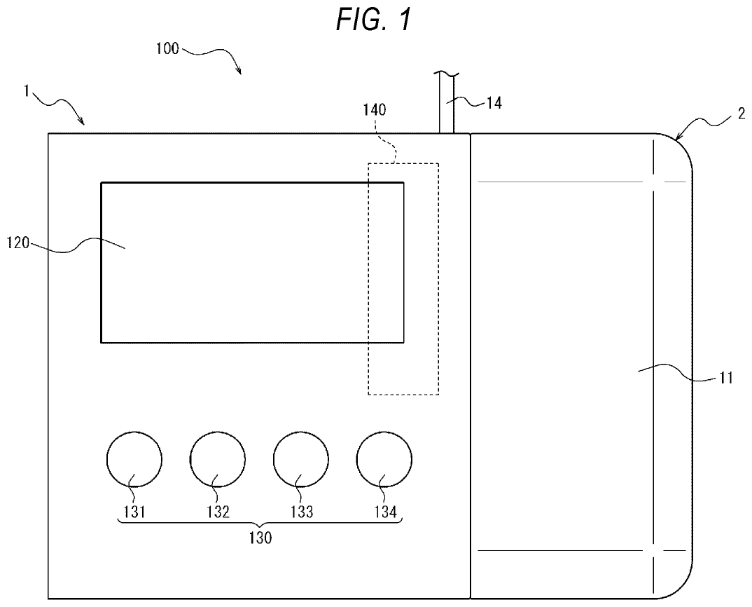

FIG. 1 is a front view illustrating an infusion pump 100 according to one embodiment of an infusion pump of the present disclosure. As shown in FIG. 1, the infusion pump 100 includes a pump main body 1 and an infusion cartridge 2 according to one embodiment of an infusion cartridge of the present disclosure. The infusion pump 100 shown in FIG. 1 can be used as, for example, a patient controlled analgesia (PCA) pump, and a use application is not particularly limited, and the infusion pump 100 may be used as a syringe pump, an infusion pump, a nutrient pump, a blood pump, or an insulin pump. Generally, an infusion pump is used for feeding an infusion in a reservoir bag by pressing an infusion tube connected to the reservoir bag outside the pump. The infusion pump 100 of the present embodiment is a PCA pump in which the pump main body 1 can be reused by replacing the disposable infusion cartridge 2.

As shown in FIG. 1, the pump main body 1 is provided with, on a front surface, a display unit 120 on which various kinds of information are displayed and an operation unit 130 on which operation switches are arranged. The display unit 120 displays, for example, a liquid feed speed and a cumulative dose. In addition, the display unit 120 may be a liquid crystal screen with a touch panel for setting the liquid feed speed or the like. The operation switches of the operation unit 130 shown in FIG. 1 are a fast forward switch 131 capable of feeding liquid at a liquid feed speed higher than the set liquid feed speed (mL/h) while being pressed, a start switch 132 for starting liquid feeding by being pressed, a stop switch 133 for forcibly stopping the liquid feeding by being pressed, and a power supply switch 134 for instructing ON/OFF of a power supply of the pump main body 1. There may be other operation switches. Accordingly, it is possible to feed an infusion into a living body, such as a patient, while controlling a flow rate.

Similar to other infusion pumps, the pump main body 1 includes a power supply unit, a storage unit, a control unit, the display unit 120, the operation unit 130, a pressing portion that is a plurality of fingers that press the infusion tube 14, a drive unit that drives these fingers, and a bubble detection sensor unit, an occlusion sensor unit, and a notification unit, such as an alarm. The pump main body 1 is not limited to the configuration of the present embodiment. The pump main body 1 may include, for example, a portion different from the above-described portions, and may be replaced with a portion having the same function.

The infusion cartridge 2 includes an accommodation portion 11 as an external case capable of accommodating a reservoir bag. A reservoir bag is accommodated in the accommodation portion 11. The reservoir bag can accommodate an infusion inside. The reservoir bag is attached with the infusion tube 14 for supplying the infusion from the inside of the reservoir bag.

As shown in FIG. 1, the infusion pump 100 is used in a state in which the infusion cartridge 2 accommodating the infusion in the reservoir bag is attached to the pump main body 1. The infusion cartridge 2 is attached to the pump main body 1 such that the infusion tube 14 is sandwiched between the pump main body 1 and the infusion cartridge 2. At this time, a pressure plate 20 to be described later is attached to a facing portion of the accommodation portion 11 of the infusion cartridge 2 that faces the pump main body 1. That is, when the pressure plate 20 is attached to the facing portion of the accommodation portion 11 in a state in which the reservoir bag accommodating the infusion is accommodated in the accommodation portion 11, the state in which the reservoir bag is accommodated in the accommodation portion 11 is maintained. At this time, a downstream side of the infusion tube 14 is positioned outside the accommodation portion 11 and is connected to a living body, such as a patient, for administration. The infusion pump 100 is implemented by attaching the infusion cartridge 2 to the pump main body 1 such that the infusion tube 14 is sandwiched between the pump main body 1 and the infusion cartridge 2.

The pump main body 1 includes a liquid feed unit 140 that feeds the infusion in the infusion tube 14 sandwiched between the pump main body 1 and the infusion cartridge 2 from a flow path upstream side (that is, a reservoir bag side) to a flow path downstream side (that is, an opposite side of the reservoir bag). The liquid feed unit 140 of the present embodiment includes the plurality of fingers serving as the pressing portion that presses the infusion tube 14, and the drive unit that drives the fingers. The plurality of fingers are disposed on a top surface of the pump main body 1 facing the pressure plate 20 positioned on a top surface of the infusion cartridge 2 facing the pump main body 1. The plurality of fingers are arranged along an extending direction of the sandwiched infusion tube 14. Each finger is driven by the drive unit so as to reciprocate in a direction facing the pressure plate 20 of the infusion cartridge 2. As each finger moves closer to the infusion cartridge 2, the infusion tube 14 is sandwiched between each finger and the pressure plate 20. Accordingly, the infusion tube 14 is occluded. The drive unit sequentially drives the fingers from the flow path upstream side toward the flow path downstream side in the extending direction of the infusion tube 14. Accordingly, the infusion tube 14 is sequentially occluded from the flow path upstream side toward the flow path downstream side, and performs peristaltic movement. Therefore, the infusion in the infusion tube 14 can be fed from the flow path upstream side toward the flow path downstream side.

As described above, the liquid feed unit 140 of the pump main body 1 of the present embodiment is implemented by pressing the infusion tube 14 with the plurality of fingers, and as long as the infusion in the infusion tube 14 can be fed, a pressing portion different from the fingers may be used. The liquid feed unit 140 of the pump main body 1 may be any pump capable of feeding the infusion to the infusion tube 14, and may be a syringe pump, a piston pump, a plunger pump, a diaphragm pump, and the like.

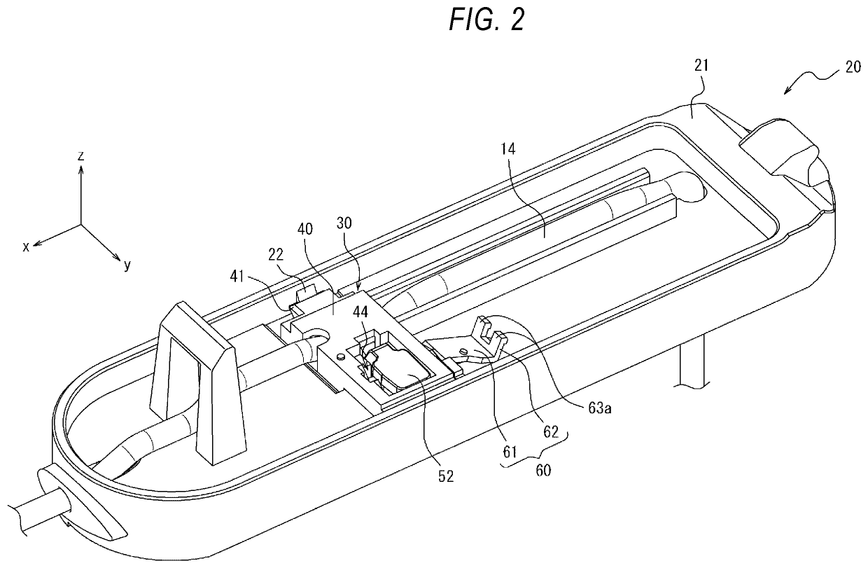

FIG. 2 is a perspective view illustrating the pressure plate 20 according to one embodiment of the present disclosure. As shown in FIG. 2, the pressure plate 20 includes a flow stop assembly 30 and a main body portion 21 to which the flow stop assembly 30 is attached. In FIG. 2, in a state in which the infusion cartridge 2 is attached to the pump main body 1, a side facing the pump main body 1 is defined as a positive z-axis direction. Therefore, in a state in which the pressure plate 20 is attached to the accommodation portion 11, the accommodation portion 11 is positioned in a negative z-axis direction of the pressure plate 20. In addition, in FIG. 2, in the state in which the infusion cartridge 2 is attached to the pump main body 1, a direction in which the infusion tube 14 extends is defined as an x-axis direction, and a direction orthogonal to an xz plane is defined as a y-axis direction.

In the state in which the infusion cartridge 2 is attached to the pump main body 1, as shown in FIG. 2, the infusion tube 14 extends in the x-axis direction and is sandwiched between the pressure plate 20 of the infusion cartridge 2 and the fingers of the pump main body 1. At this time, as shown in FIG. 2, a part of the infusion tube 14 extending in the x-axis direction is held by the flow stop assembly 30. The flow stop assembly 30 is attached to the pressure plate 20 in an assembled state. Here, the assembled state of the flow stop assembly 30 refers to a state in which a tube receiving portion 40 and a tube pressing portion 50 to be described later are disposed such that the infusion tube 14 can be pressed by the tube receiving portion 40 and the tube pressing portion 50. In the present embodiment, the assembled state of the flow stop assembly 30 particularly refers to a state in which the tube pressing portion 50 is rotationally displaced with a hinge 31 as a rotation axis such that the tube receiving portion 40 and the tube pressing portion 50 face each other in a state in which the infusion tube 14 is held by the tube receiving portion 40.

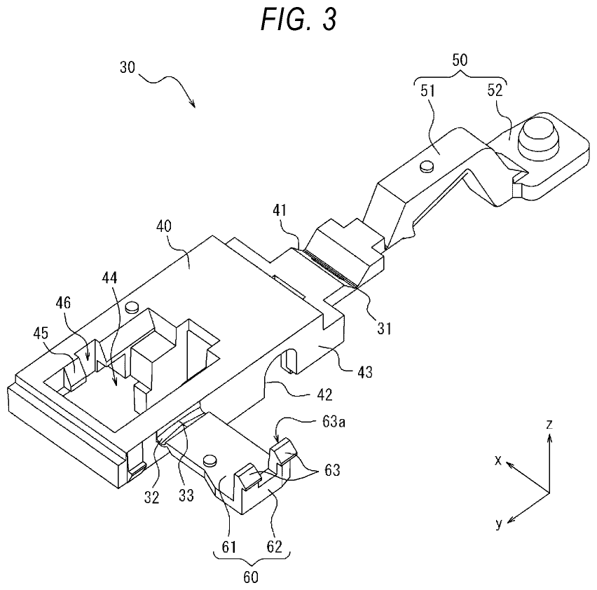

FIG. 3 is a perspective view illustrating the flow stop assembly 30 before assembly according to one embodiment of the present disclosure. As shown in FIG. 3, the flow stop assembly 30 includes the tube receiving portion 40, the tube pressing portion 50, and a release clip 60. An x-axis direction, a y-axis direction, and a z-axis direction in FIG. 3 are the same as the x-axis direction, the y-axis direction, and the z-axis direction in FIG. 2, respectively, with respect to the tube receiving portion 40. That is, the x-axis directions, the y-axis directions, and the z-axis directions in FIGS. 2 and 3 are the same as each other when viewed from the tube receiving portion 40.

In the present embodiment, the tube receiving portion 40 and the tube pressing portion 50 are coupled to each other via the hinge 31. In the present embodiment, before assembly, the tube pressing portion 50 is coupled to the tube receiving portion 40 so as to be positioned on a y-axis negative direction side of the tube receiving portion 40. In addition, in the present embodiment, the tube receiving portion 40 and the release clip 60 are coupled to each other via a hinge 32. In the present embodiment, before assembly, the release clip 60 is coupled to the tube receiving portion 40 so as to be positioned on an negative x-axis direction side of the tube receiving portion 40. Therefore, the tube pressing portion 50 and the release clip 60 are disposed at orthogonal positions with respect to the tube receiving portion 40. A positional relation between the tube pressing portion 50 and the release clip 60 is not limited thereto. The release clip 60 may be disposed at a position facing the tube pressing portion 50 or may be disposed adjacent to the tube pressing portion 50.

The tube receiving portion 40 includes a fixing portion 41 for fixing the flow stop assembly 30 to the infusion pump 100. In other words, the flow stop assembly 30 is attached to the infusion pump 100 by fixing the fixing portion 41 to the infusion pump 100. The fixing portion 41 may be formed in any form in which the flow stop assembly 30 is fixable to the infusion pump 100. In the present embodiment, the fixing portion 41 is particularly fixable to the main body portion 21 of the pressure plate 20. Specifically, the main body portion 21 of the pressure plate 20 may include an engagement claw 22, and the fixing portion 41 may be implemented by a convex portion that can be engaged with the engagement claw 22. A form of the fixing portion 41 is not limited thereto, and a plurality of fixing portions 41 may be provided.

The tube receiving portion 40 includes a restriction portion 42 that restricts displacement of the infusion tube 14. The restriction portion 42 may restrict the displacement in at least a part of the directions other than the extending direction (that is, the x-axis direction) of the infusion tube 14 in the state in which the flow stop assembly 30 is assembled. In the present embodiment, as shown in FIG. 3, the restriction portion 42 is implemented by a groove provided in a wall surface 43 of the tube receiving portion 40. When the flow stop assembly 30 is assembled, the infusion tube 14 is inserted and disposed in the restriction portion 42 implemented by the groove, thereby restricting the displacement of the infusion tube 14 in the y-axis direction and the positive z-axis direction. Because the restriction portion 42 restricts the displacement of the infusion tube 14, the holding state of the infusion tube 14 by the flow stop assembly 30 is easily stabilized.

The tube receiving portion 40 has a through hole 44 penetrating in the z-axis direction. As will be described later in detail, the through hole 44 is a hole through which a head portion 52 of the tube pressing portion 50 is inserted in the state in which the flow stop assembly 30 is assembled.

As shown in FIG. 3, the tube pressing portion 50 includes an arm portion 51 extending from the hinge 31 toward the negative y-axis direction, and the head portion 52 positioned on a tip end of the arm portion 51, that is, at the other end opposite to the hinge 31.

The tube pressing portion 50 is rotationally displaceable with respect to the tube receiving portion 40 with the hinge 31 as an axis. That is, in the example shown in FIG. 3, the tube pressing portion 50 is rotationally displaceable with respect to the tube receiving portion 40 with an axis parallel to an x axis along the hinge 31 (hereinafter, also simply referred to as “axis of the hinge 31”) as a rotation axis. Specifically, when viewed in the negative x-axis direction, the tube pressing portion 50 is rotationally displaceable in a clockwise direction with the axis of the hinge 31 as the rotation axis. When the flow stop assembly 30 is assembled, in a state in which the infusion tube 14 is disposed in the restriction portion 42 of the tube receiving portion 40, the tube pressing portion 50 is rotationally displaced clockwise with the axis of the hinge 31 as the rotation axis when viewed from the negative x-axis direction, and the head portion 52 is positioned in the through hole 44 as shown in FIG. 2. Accordingly, the infusion tube 14 is inserted between the restriction portion 42 of the tube receiving portion 40 and the arm portion 51 of the tube pressing portion 50. As described above, in the state in which the infusion tube 14 is inserted between the tube receiving portion 40 and the tube pressing portion 50, the flow stop assembly 30 is fixed to the main body portion 21 of the pressure plate 20 by the fixing portion 41.

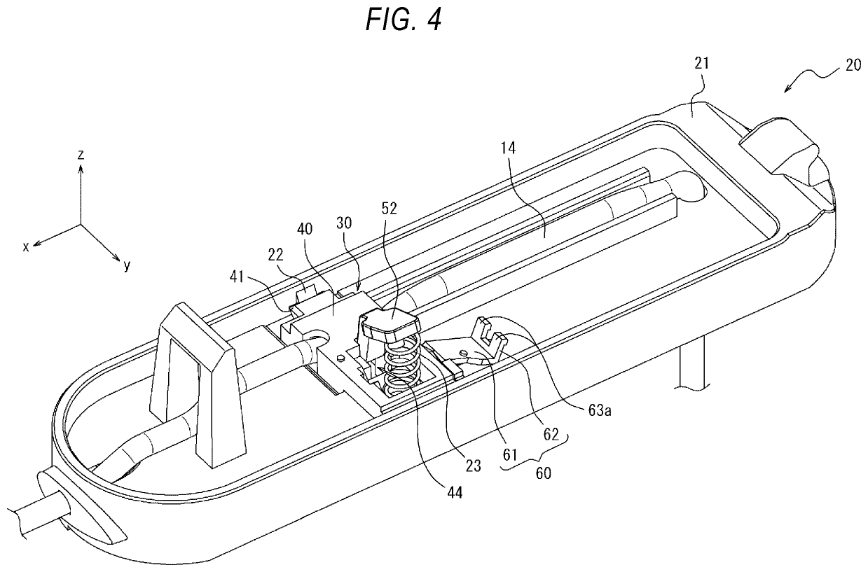

In a state in which the flow stop assembly 30 is assembled and attached to the main body portion 21 of the pressure plate 20, the head portion 52 of the tube pressing portion 50 is biased in a direction to block the infusion tube 14. In the present embodiment, as shown in FIG. 2, the head portion 52 of the tube pressing portion 50 is biased in the positive z-axis direction. The biasing may be performed by using any method. For example, the head portion 52 may be biased in the positive z-axis direction by an elastic force of an elastic body 23 (see FIG. 4), such as a spring provided on the pressure plate 20.

By being biased in this manner, the tube pressing portion 50 can move toward the tube receiving portion 40 and press the infusion tube 14 in the state in which the infusion tube 14 is inserted between the tube pressing portion 50 and the tube receiving portion 40. Specifically, when the head portion 52 is biased in the positive z-axis direction, as shown in FIG. 4, the head portion 52 of the tube pressing portion 50 protrudes from the through hole 44 in the positive z-axis direction. In this state, the infusion tube 14 is pressed and blocked by the arm portion 51. On the other hand, when the head portion 52 is pushed down from the positive z-axis direction to the negative z-axis direction and the head portion 52 is positioned in the through hole 44 as shown in FIG. 2, the infusion tube 14 is not pressed and is not blocked by the arm portion 51. As described above, the tube pressing portion 50 can selectively block the infusion tube 14 in a state in which the fixing portion 41 is fixed to the main body portion 21 of the pressure plate 20 of the infusion pump 100. Here, the term “selectively block” means that the tube pressing portion 50 is movable relative to the tube receiving portion 40 between a blocking permission position at which blocking of the infusion tube 14 between the tube pressing portion 50 and the tube receiving portion 40 is permitted and a blocking release position at which the infusion tube 14 is not blocked between the tube pressing portion 50 and the tube receiving portion 40.

The pump main body 1 includes a push-down portion, such as a convex portion, that pushes down the head portion 52 in the negative z-axis direction when the infusion cartridge 2 is attached. Accordingly, in the state in which the infusion cartridge 2 is attached to the pump main body 1, the head portion 52 is pushed down in the negative z-axis direction and is positioned at a position shown in FIG. 2. Accordingly, in the state in which the infusion cartridge 2 is attached to the pump main body 1, the infusion tube 14 is not blocked.

When the infusion cartridge 2 is detached from the pump main body 1, the head portion 52 is displaced in the positive z-axis direction by, for example, a biasing force of the elastic body 23, and moves to a position shown in FIG. 4. Accordingly, in a state in which the infusion cartridge 2 is not attached to the pump main body 1, the infusion tube 14 is blocked. In this way, the flow stop assembly 30 functions as an AFF mechanism.

Referring again to FIG. 3, the release clip 60 includes a main body portion 61 and an end portion 62 positioned on a tip end of the main body portion 61, that is, the other end opposite to the hinge 32.

The release clip 60 is rotationally displaceable with respect to the tube receiving portion 40 with the hinge 32 as an axis. That is, in the example shown in FIG. 3, the release clip 60 is rotationally displaceable with respect to the tube receiving portion 40 with an axis parallel to a y axis along the hinge 32 (hereinafter, also simply referred to as “axis of the hinge 32”) as a rotation axis. Specifically, when viewed from a positive y-axis direction, the release clip 60 is rotationally displaceable in a counterclockwise direction with the axis of the hinge 32 as the rotation axis. Specifically, the release clip 60 is movable between the blocking permission position and the blocking release position by rotational displacement around the hinge 32. The blocking permission position is a position at which the movement of the tube pressing portion 50 toward a tube receiving portion 40 side is permitted and the infusion tube 14 can be blocked between the tube pressing portion 50 and the tube receiving portion 40. In other words, the blocking permission position is a position at which the release clip 60 does not interfere with the tube pressing portion 50. On the other hand, the blocking release position is a position at which the release clip 60 comes into contact with the tube pressing portion 50 to restrict the movement of the tube pressing portion 50 toward the tube receiving portion 40 side. That is, the blocking release position is a position at which the release clip 60 interferes with the tube pressing portion 50. Accordingly, the infusion tube 14 cannot be blocked between the tube pressing portion 50 and the tube receiving portion 40. The blocking permission position and the blocking release position will be further described below.

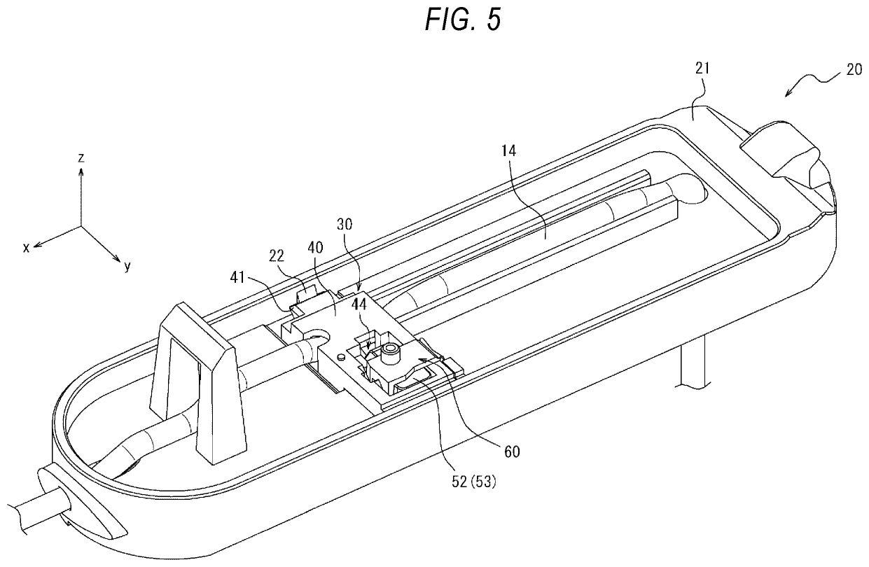

In a state in which the flow stop assembly 30 is attached to the main body portion 21 of the pressure plate 20 and is in the blocking permission position, the main body portion 61 comes into contact with the head portion 52 of the tube pressing portion 50 by rotationally displacing the release clip 60 counterclockwise as viewed from the positive y-axis direction. In this state, by displacing the tube pressing portion 50 in a direction opposite to a direction in which the tube pressing portion 50 is biased by the elastic body 23, that is, in a direction away from the tube receiving portion 40, the release clip 60 can move to the blocking release position and can release a blocked state of the infusion tube 14. Specifically, in the present embodiment, when the release clip 60 is further rotationally displaced counterclockwise as viewed from the positive y-axis direction from the state in which the main body portion 61 is in contact with the head portion 52, that is, when the release clip 60 is pushed down in the negative z-axis direction, the head portion 52 is pushed down in the negative z-axis direction as shown in FIG. 5. Accordingly, the arm portion 51 is also pushed down in the negative z-axis direction, and as a result, a state in which the infusion tube 14 is blocked by the tube receiving portion 40 and the arm portion 51 is released. In this way, by rotationally displacing the release clip 60, the release clip 60 can be moved to the blocking release position to release the blocked state of the infusion tube 14. In the state in which the infusion cartridge 2 is not attached to the pump main body 1, the blocked state of the infusion tube 14 can be released by rotationally displacing the release clip 60 as described above. In the state in which the infusion cartridge 2 is not attached to the pump main body 1, operations such as filling the reservoir bag with the infusion can be performed by releasing the blocked state of the infusion tube 14.

As shown in FIG. 3, the release clip 60 includes a hook portion 63 at the end portion 62. In addition, the tube receiving portion 40 includes an engagement portion 45 to be engaged with the hook portion 63. The hook portion 63 of the release clip 60 and the engagement portion 45 can be engaged with each other in a state in which the tube pressing portion 50 is displaced in the direction away from the tube receiving portion 40. More specifically, in the present embodiment, the hook portion 63 and the engagement portion 45 can be engaged with each other in a state in which the head portion 52 of the tube pressing portion 50 is pressed down in the negative z-axis direction by the release clip 60 and the blocked state of the infusion tube 14 is released. By engaging the hook portion 63 with the engagement portion 45, the state in which the head portion 52 is pushed down by the release clip 60 can be maintained without continuous pressing by, for example, a finger. That is, the release clip 60 can remain at the blocking release position. The engagement portion 45 is provided at a position at which the engagement portion 45 can engage with the hook portion 63 in the state in which the release clip 60 displaces the tube pressing portion 50 as described above.

It is preferred that the tube pressing portion 50 is positioned on a back surface 63a side at which the hinge 32 is positioned with respect to the hook portion 63 in the state in which the hook portion 63 and the engagement portion 45 are engaged with each other. That is, in the present embodiment, it is preferred that the head portion 52 of the tube pressing portion 50 is pushed down in the negative z-axis direction by the release clip 60, and the head portion 52 is positioned on the back surface 63a side of the hook portion 63 in the state in which the hook portion 63 and the engagement portion 45 are engaged with each other. At this time, the shorter a distance between the back surface 63a and the head portion 52, the more preferable. As described above, when the head portion 52 is positioned on the back surface 63a side of the hook portion 63, it is possible to prevent the hook portion 63 from being displaced in the negative x-axis direction. In particular, in a case in which the release clip 60 is implemented by a member having elasticity, a center of the main body portion 61 of the release clip 60 is pressed in the positive z-axis direction by the biasing force in the positive z-axis direction with respect to the head portion 52, and the pressed portion is curved and deformed in the positive z-axis direction, and thus the end portion 62 and the hook portion 63 are easily displaced in the negative x-axis direction side. Accordingly, when the displacement is greater than or equal to a predetermined value, an engagement state between the hook portion 63 and the engagement portion 45 may be unintentionally released. However, because the head portion 52 is positioned on the back surface 63a side of the hook portion 63, the displacement of the hook portion 63 in the negative x-axis direction can be prevented. In other words, the tube pressing portion 50 includes a restriction portion 53 capable of restricting the deformation or movement of the hook portion 63 on a side opposite to the engagement portion 45 with the hook portion 63 interposed therebetween. As shown in FIG. 5, the restriction portion 53 of the present embodiment is implemented by the head portion 52. In the present embodiment, the displacement of the hook portion 63 in the negative x-axis direction can be restricted by the head portion 52. As a result, it is possible to prevent the engagement state between the hook portion 63 and the engagement portion 45 from being unintentionally released.

The tube receiving portion 40 and the release clip 60 may be torsionally displaced relative to each other in a rotation axis direction of the hinge 32 (y-axis direction in the present embodiment). The hinge 32 may be implemented by, for example, a material having flexibility or elasticity so as to be torsionally displaceable. The hinge 32 may have other mechanisms that allow it to be torsionally displaceable, for example. In addition, in order to prevent deformation of the flow stop assembly 30 and displacement caused by the deformation of the flow stop assembly 30, the tube receiving portion 40, the tube pressing portion 50, and the release clip 60 may have a reinforcing structure. The reinforcing structure may be appropriately selected based on various methods including, for example, molding a plurality of types of resins in combination, molding a portion that is easily deformed to be thick, and bonding a separate member, such as a metal plate.

For example, the hinge 32 may be formed such that a length of a member forming the hinge 32 in the x-axis direction increases as a distance from a point (hereinafter, referred to as a specific point) 33 of the hinge 32 along the y-axis direction increases. Accordingly, the length of the member forming the hinge 32 in the x-axis direction at a position in which the hinge 32 is separated from the specific point 33 along the y-axis direction is greater than the length of the member forming the hinge 32 in the x-axis direction at the specific point 33. Therefore, the length of the member forming the hinge 32 increases and a torsion width increases as the distance from the specific point 33 along the y-axis direction increases. In this way, the release clip 60 may be torsionally displaced in the axis direction of the hinge 32 with respect to the tube receiving portion 40.

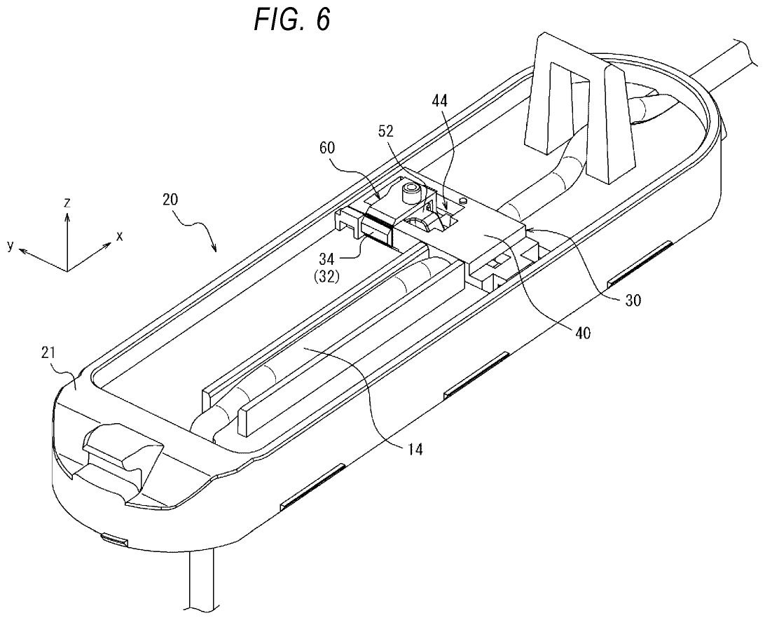

As another example, as shown in FIG. 6, the hinge 32 may be implemented by an elastic member 34 having a predetermined length or more in a coupling direction (x-axis direction in a state before assembly as shown in FIG. 3) in which the tube receiving portion 40 and the release clip 60 are coupled to each other. In this example, the length of the hinge 32 may be uniform along the y-axis direction. In this example, the elastic member 34 itself as the hinge 32 is torsionally deformed as a whole, and thus the end portion 62 of the release clip 60 is torsionally displaceable within a predetermined displacement width. In other words, the elastic member 34 has a length greater than or equal to a predetermined length that allows the above-described torsional deformation. In this way, the release clip 60 may be torsionally displaced.

The tube receiving portion 40 includes a release portion 46 capable of releasing the engagement state between the hook portion 63 and the engagement portion 45 in the state in which the end portion 62 is torsionally displaced. That is, the release portion 46 releases the engagement state when the end portion 62 is torsionally displaced. In the example shown in FIG. 3, the release portion 46 is implemented by a concave portion provided adjacent to the engagement portion 45 on a wall surface provided with the engagement portion 45. Accordingly, when the end portion 62 is torsionally displaced, the engagement state between the hook portion 63 and the engagement portion 45 is released, and the hook portion 63 can be pulled out from the release portion 46 implemented by the concave portion by rotationally displacing the release clip 60 clockwise when viewed from the positive y-axis direction. As described above, because the release portion 46 is provided, even when the head portion 52 is positioned on the back surface 63a side of the hook portion 63 as described above, the engagement state can be released by twisting the release clip 60.

Next, a method of assembling the infusion cartridge 2 including a method of assembling the flow stop assembly 30 will be described.

First, the reservoir bag, the accommodation portion 11, and the main body portion 21 and the flow stop assembly 30 forming the pressure plate 20 are prepared. The flow stop assembly 30 is the flow stop assembly 30 shown in FIG. 3 described in the above-described embodiment.

Then, the flow stop assembly 30 is assembled. Specifically, in the present embodiment, the infusion tube 14 is disposed to insert through the restriction portion 42, and the tube pressing portion 50 is rotationally displaced clockwise when viewed from the negative x-axis direction with the x axis as a rotation axis. Accordingly, the infusion tube 14 is inserted between the tube receiving portion 40 and the tube pressing portion 50. The flow stop assembly 30 assembled in this way is attached to the main body portion 21. Accordingly, the pressure plate 20 including the main body portion 21 and the flow stop assembly 30 is formed. In the pressure plate 20, the head portion 52 of the tube pressing portion 50 is biased in the positive z-axis direction by the elastic body 23.

Finally, the reservoir bag is accommodated in the accommodation portion 11, and the pressure plate 20 including the flow stop assembly 30 is attached to a top surface of the accommodation portion 11. In this way, the infusion cartridge 2 is assembled.

Here, the release clip 60 is disposed at the blocking permission position when the infusion cartridge 2 is assembled. After the infusion cartridge 2 is assembled, the blocked state of the infusion tube 14 is released by moving the release clip 60 to the blocking release position.

As described above, in the flow stop assembly 30 according to the present embodiment, the tube receiving portion 40, the tube pressing portion 50, and the release clip 60 are coupled to one another by the hinge 31 and the hinge 32 to form a single member. Therefore, as compared with a case in which the tube receiving portion 40, the tube pressing portion 50, and the release clip 60 are implemented by independent members, the number of components forming the flow stop assembly 30 can be reduced. Because the number of components to be manufactured is reduced by reducing the number of components, a manufacturing cost is easily reduced. In addition, by reducing the number of components, it is not necessary to assemble the flow stop assembly 30 by combining a plurality of components, and thus the assembly is simplified, and it is possible to reduce the occurrence of failure due to, for example, forgetting to attach a component.

In particular, in the present embodiment, because the tube receiving portion 40 and the tube pressing portion 50 are formed as a single member, the infusion tube 14 can be inserted into between the tube receiving portion 40 and the tube pressing portion 50 by rotationally displacing the tube pressing portion 50, so that the assembly is easy, and a possibility of occurrence of positional deviation during assembly is reduced. In addition, because it is not necessary to combine a plurality of members, a speed of assembly is increased.

In addition, in the present embodiment, the tube receiving portion 40 and the release clip 60 are formed as a single member, and thus the release clip 60 is prevented from being lost.

In the above-described embodiments, the example in which the tube receiving portion 40, the tube pressing portion 50, and the release clip 60 are formed as a single member is described, but the flow stop assembly 30 according to the present disclosure does not necessarily have to be implemented such that three constituent elements are formed as a single member. In the present disclosure, at least, the tube receiving portion 40 and the tube pressing portion 50, or the tube receiving portion 40 and the release clip 60 may be formed as a single member. At least the tube receiving portion 40 and the tube pressing portion 50, or the tube receiving portion 40 and the release clip 60 are formed as a single member, so that the number of components can be reduced. In addition, in the above-described embodiments, the hinge 32 is torsionally displaceable, but the hinge 32 need not be formed to be torsionally displaceable. Further, in the above-described embodiments, the engagement portion 45 is provided on an inner wall surface of the through hole 44. However, the position of the engagement portion 45 is not particularly limited. The engagement portion 45 may be provided on a wall surface of the tube receiving portion 40 (which is different from the inner wall surface of the through hole 44), such as an outer wall surface at the positive x-axis direction side of the tube receiving portion 40. In other words, the position of the engagement portion 45 may be appropriately set according to the position of the hook portion 63. In addition, in the above-described embodiments, it is described that the hook portion 63 having a hook-shape is hooked to the engagement portion 45 to engage with each other, but it is not limited to this engagement configuration. The tube receiving portion 40 and the release clip 60 may be formed to engage with each other, for example, by fitting a convex portion provided on one of the receiving portion 40 and the release clip 60 into a concave portion or a hole portion provided on the other.

The flow stop assembly, the pressure plate, the infusion cartridge, and the method of assembling an infusion cartridge according to the present disclosure are not limited to the specific configurations and steps described in the above-described embodiments, and various modifications and changes are made without departing from the scope of the claims.

The present disclosure relates to a flow stop assembly, a pressure plate, an infusion cartridge, and a method of assembling an infusion cartridge.

### REFERENCE NUMERAL LIST

1: pump main body
2: infusion cartridge
11: accommodation portion
14: infusion tube
20: pressure plate
21: main body portion
22: engagement claw
23: elastic body
30: flow stop assembly
31, 32: hinge
33: specific point
34: elastic member
40: tube receiving portion
41: fixing portion
42: restriction portion
43: wall surface
44: through hole
45: engagement portion
46: release portion
50: tube pressing portion
51: arm portion
52: head portion
53: restriction portion
60: release clip
61: main body portion
62: end portion
63: hook portion
63a: back surface
100: infusion pump
120: display unit
130: operation unit
131: fast forward switch
132: start switch
133: stop switch
134: power supply switch
140: liquid feed unit

1. A flow stop assembly attachable to an infusion pump, the flow stop assembly comprising:
a tube receiving portion comprising a fixing portion configured to be fixed to the infusion pump; and
a tube pressing portion coupled to the tube receiving portion via a hinge and rotationally displaceable with respect to the tube receiving portion around a rotation axis, wherein:
the fixing portion is fixable to the infusion pump in a state in which an infusion tube is inserted between the tube receiving portion and the tube pressing portion.

2. The flow stop assembly according to claim 1, wherein:
the tube pressing portion is biased so as to selectively block the infusion tube in a state in which the fixing portion is fixed to the infusion pump.

3. The flow stop assembly according to claim 1, wherein:
the tube receiving portion comprises a restriction portion configured to restrict displacement of the infusion tube.

4. The flow stop assembly according to claim 3, wherein:
the restriction portion comprises a groove formed in the tube receiving portion.

5. A pressure plate of an infusion pump, the pressure plate comprising:
a flow stop assembly; and
a main body portion to which the flow stop assembly is attached, wherein:
the flow stop assembly comprises a tube receiving portion comprising a fixing portion configured to be fixed to the main body portion, and a tube pressing portion coupled to the tube receiving portion via a hinge and rotationally displaceable with respect to the tube receiving portion around a rotation axis, and
the fixing portion is fixed to the main body portion in a state in which an infusion tube is inserted between the tube receiving portion and the tube pressing portion.

6. An infusion cartridge for an infusion pump, the infusion cartridge comprising:
an accommodation portion configured to accommodate a reservoir bag accommodating an infusion;
an infusion tube configured to supply the infusion from the reservoir bag; and
a pressure plate attached to the accommodation portion, wherein:
the pressure plate comprises a flow stop assembly,
the flow stop assembly comprises a tube receiving portion, and a tube pressing portion coupled to the tube receiving portion via a hinge and rotationally displaceable with respect to the tube receiving portion around a rotation axis, and
the infusion tube is inserted between the tube receiving portion and the tube pressing portion.

7. A method of assembling an infusion cartridge of an infusion pump, the method comprising:
a step of preparing:
a reservoir bag configured to accommodate an infusion,
an accommodation portion configured to accommodate the reservoir bag, and
a main body portion and a flow stop assembly forming a pressure plate, wherein:
the flow stop assembly comprises a tube receiving portion, and a tube pressing portion coupled to the tube receiving portion via a hinge and rotationally displaceable with respect to the tube receiving portion around a rotation axis;
a step of attaching the flow stop assembly to the main body portion in a state in which an infusion tube connected to the reservoir bag and configured to supply the infusion is inserted between the tube receiving portion and the tube pressing portion; and
a step of attaching the pressure plate to the accommodation portion.

8. A flow stop assembly attachable to an infusion pump, the flow stop assembly comprising:
a tube receiving portion comprising a fixing portion configured to be fixed to the infusion pump;
a tube pressing portion configured to press an infusion tube by moving toward the tube receiving portion in a state in which the infusion tube is inserted between the tube receiving portion and the tube pressing portion; and
a release clip coupled to the tube receiving portion via a hinge and rotationally displaceable with respect to the tube receiving portion around a rotation axis, wherein:
the release clip is configured to release, by the rotational displacement, a blocked state of the infusion tube by displacing the tube pressing portion in a direction away from the tube receiving portion.

9. The flow stop assembly according to claim 8, wherein:
the tube receiving portion and an end portion of the release clip are displaceable relative to each other in a direction parallel to the rotation axis.

10. The flow stop assembly according to claim 8, wherein:
the release clip comprises a hook portion at an end portion not coupled to the hinge, and
the tube receiving portion comprises an engagement portion configured to engage with the hook portion in a state in which the release clip displaces the tube pressing portion in the direction away from the tube receiving portion.

11. The flow stop assembly according to claim 10, wherein:
the tube receiving portion comprises a release portion configured to release an engagement state between the hook portion and the engagement portion in a state in which the end portion of the release clip is displaced with respect to the tube receiving portion in the direction parallel to the rotation axis.

12. The flow stop assembly according to claim 10, wherein:
the tube pressing portion comprises a restriction portion configured to restrict deformation or movement of the hook portion on a side opposite to the engagement portion with the hook portion interposed therebetween in a state in which the hook portion and the engagement portion are engaged with each other.

13. An infusion cartridge of an infusion pump, the infusion cartridge comprising:
an accommodation portion configured to accommodate a reservoir bag accommodating an infusion;
an infusion tube configured to supply the infusion from the reservoir bag; and
a pressure plate attached to the accommodation portion, wherein:
the pressure plate comprises a flow stop assembly,
the flow stop assembly comprises:
a tube receiving portion,
a tube pressing portion configured to press the infusion tube by moving toward the tube receiving portion in a state in which the infusion tube is inserted between the tube receiving portion and the tube pressing portion, and
a release clip coupled to the tube receiving portion via a hinge and rotationally displaceable with respect to the tube receiving portion around a rotation axis, and
the release clip is configured to release, by the rotational displacement, a blocked state of the infusion tube by displacing the tube pressing portion in a direction away from the tube receiving portion.
